(19) United States
(12) Patent Application Publication
(10) Pub. No.: US 2026/0132108 A1
(43) Pub. Date: May 14, 2026

(54) PREPARATION OF MESEMBRINE

(71) Applicant: Kanna Health Limited, Kent (GB)

(72) Inventor: Ryan Joseph PROTZKO, Marriotsville, MD (US)

(21) Appl. No.: 19/118,608

(22) PCT Filed: Oct. 6, 2023
(86) PCT No.: PCT/GB2023/052585

§ 371 Date: Apr. 4, 2025

(30) Foreign Application Priority Data
Oct. 7, 2022 (GB) 2214772.2

(51) Int. Cl. C07D 209/12; A61K 31/404

(52) U.S. Cl. CPC C07D 209/12; A61K 31/404

(57) ABSTRACT

The present invention provides an improved method for the preparation of mesembrine. In a further embodiment the present invention relates to the preparation of enantiopure or enantioenriched mesembrine.

### FIELD OF THE INVENTION

The present invention provides an improved method for the preparation of mesembrine. In a further embodiment the present invention relates to the preparation of enantiopure or enantioenriched mesembrine.

Mesembrine (3a-(3,4-dimethoxyphenyl)-octahydro-1-methy-6H-indol-6-one), is an alkaloid which naturally occurs in the Sceletium tortuosum species of plant. Although the mesembrine molecule can have four different diastereomers the plant only produces the laevorotary isomer (3aS, 7aS)-3a-(3,4-dimethoxyphenyl)-1-methyloctahydro-6H-indol-6-one or (S,S)-mesembrine. Synthetically prepared mesembrine produces a mixture of both the (S—S)-mesembrine isomer and (3aR, 7aR)-3a-(3,4-dimethoxyphenyl)-1-methyloctahydro-6H-indol-6-one or (R,R)-mesembrine.

Due to the lack of stereoselectivity of the synthetic method, in order to prepare an enantiopure or an enantioenriched form of either (S, S)-mesembrine or (R,R)-mesembrine an additional step using chiral column separation is required. Such a step is not suitable for commercial production of either isomer as it is slow and poorly yielding. The present invention provides a chiral resolution method which is economically efficient and enables production of commercial scale quantities of enantiopure or enantioenriched mesembrine.

### BACKGROUND TO THE INVENTION

Mesembrine is an alkaloid which naturally occurs in the Sceletium tortuosum species of plants indigenous to South Africa. The genus Sceletium, classified under the Aizoaceae family, is indigenous to the Western, Eastern and Northern Cape province of South Africa. In addition to mesembrine other alkaloids are found in extracts of Sceletium tortuosum including mesembrenol, Δ⁷mesembrenone, mesembranol, mesembrenone, and epimesembranol.

In addition, there are several other known mesembrine alkaloids found in varying quantities in extracts of S. tortuosum including, 4′-O-demethylmesembranol, 4′-O-demethylmesembrenol, 4′-O-demethylmesembrenone, sceletenone, N-demethly-N-formulmesembrenone, O-acetylmesembrenol, mesembrane, N-demethelymesembrenol, N-demethylmesembranol, hordenine, joubertiamine, dehydrojoubertiamine, dehydrojoubertiamine, joubertinamine, O-methyldehydrojoubertiamine, O-methyljoubertiamine, O-methyldihydrojoubertiamine, 3′-methoxy-4′-O-methyljoubertiamine, 3′-methoxy-4′O-methyljoubertiaminol, 4-(3,4-dimethoxyphenyl-4-[2-acetylmethylamino)ethyl]cyclohexanone, 4-(3-methoxy-4-hydroxy-phenyl)-4-[2-acetylmethylamino)ethyl]cyclohexadienone, sceletium alkaloid A-4, tortuosamine, N-formultortuosamine, and N-acetyltotuosamine.

Preparations containing extracts of S. tortuosum have a long history of use in traditional medicine by the San and Khoikhoi people in South Africa where it was used as a masticatory and a medicine to quench their thirst, fight fatigue and for healing, social, and spiritual purposes.

The structure of mesembrine was described by Popelak et al., 1960 and the configuration by P. W. Jeffs et al., 1969. Mesembrine occurs naturally as (S—S)-mesembrine.

Mesembrine can be isolated from extracts of S. tortuosum however isolation of the (S—S)-mesembrine isomer from a botanical source is not a suitable method to prepare the compound in the large quantities required for making and testing a compound as a pharmaceutical. This is due to a small amount of mesembrine being produced by the plant; about 0.3% mesembrine in the roots and 0.86% in the leaves, stems, and flowers of the plant by dry weight, therefore purification efforts are challenging and small yields of purified mesembrine would be produced.

Mesembrine can be synthesized chemically using the method described by Stevens and Wentland, 1968. This method produces a racemic mixture of the two isomers (R—R)-mesembrine and (S—S)-mesembrine. Separation of the two isomers on a commercially relevant scale has heretofore not been described. Previously column separation methods have been described at small scale for analytical purposes. However, no chiral resolution methods have been described, such methods are much more economically efficient and commercially relevant compared to chiral column separation.

Many pharmaceuticals are prepared and are marketed in one specific enantiomeric form and often biological molecules are formed in only one of many possible chiral forms. Often different enantiomers of a chiral molecule can bind differently or do not bind at all to target receptors.

It is known that whereas one enantiomer of a drug may have a desired beneficial effect while the other may cause serious and undesired side effects, an example of this is thalidomide. The (R)-thalidomide enantiomer is effective against morning sickness however the(S)-thalidomide enantiomer causes congenital abnormalities. Other medicaments are effective in their racemic form but are additionally produced in a single enantiomeric form, for example fenfluramine and dexfenfluramine.

It is often uneconomical to produce compounds as individual enantiomers as there does not exist a method suitable to produce the compounds in high enough yield and purity.

An object of the present invention is an improved method for the synthetic production of mesembrine. A further object of the present invention is the preparation of an enantiopure or enantioenriched form of mesembrine.

Surprisingly, it was found that the use of specific resolving agents were able to produce significantly enantiomerically enriched forms of mesembrine.

The advantages that the method of the present invention provides is the ability to prepare substantial quantities of either racemic, enantiopure or enantioenriched mesembrine material which can be used to formulate into medicaments or for use as test agents in clinical and non-clinical studies.

### BRIEF SUMMARY OF THE DISCLOSURE

In accordance with a first aspect of the present invention there is provided a method for the preparation of 3a-(3,4-Dimethoxyphenyl)-1-methyloctahydro-6H-indol-6-one (racemic mesembrine, compound 6) comprising the steps of:
a) Reacting NaH with 2-(3,4-dimethoxyphenyl) acetonitrile (compound 2) in DMF to produce 1-(3,4-dimethoxyphenyl)cyclopropane-1-carbonitrile (compound 3);
b) Suspending 1-(3,4-Dimethoxyphenyl)cyclopropane-1-carbonitrile (compound 3) in diethyl ether and cooling in a water bath with DIBAL in hexane to produce 1-(3,4-Dimethoxyphenyl)cyclopropane-1-carbaldehyde (compound 4);
c) Dissolving 1-(3,4-Dimethoxyphenyl)cyclopropane-1-carbaldehyde (compound 4) in THF and adding methylamine and magnesium sulfate to produce 1-(1-(3,4-Dimethoxyphenyl)cyclopropyl)-N-methylmethanimine (compound 5); and
d) Stripping 1-(1-(3,4-Dimethoxyphenyl)cyclopropyl)-N-methylmethanimine (compound 5) with toluene dissolved in DCM to prepare 3a-(3,4-Dimethoxyphenyl)-1-methyloctahydro-6H-indol-6-one (racemic mesembrine, compound 6).

In accordance with a second aspect of the present invention there is provided a method for the preparation of an enantiopure or enantioenriched preparation of (S—S)-mesembrine comprising the steps of:
a) Adding (+)-Dibenzoyl-D-tartaric acid monohydrate to a solution of racemic mesembrine in 2-propanol; and
b) Collecting the precipitate.

In accordance with a third aspect of the present invention there is provided a method for the preparation of and enantiopure preparation of (+)-mesembrine comprising the steps of:
a) Adding (+)-Dibenzoyl-D-tartaric acid monohydrate to a solution of racemic mesembrine in 2-propanol; and
b) Collecting the mother liquor and drying.

Preferably the yield of (S—S)-mesembrine is greater than 75%, more preferably greater than 80%, more preferably greater than 85%, more preferably greater than 90%, more preferably greater than 95%, or more preferably still greater than 99%.

Preferably the yield of (R—R)-mesembrine is greater than 75%, more preferably greater than 80%, more preferably greater than 85%, more preferably greater than 90%, more preferably greater than 95%, or more preferably still greater than 99%.

Preferably the amount of (S—S)-mesembrine and/or (R—R)-mesembrine produced using the method of the invention is greater than 50 g, more preferably greater than 100 g, more preferably greater than 500 g, more preferably greater than 1 kg, more preferably greater than 5 kg, more preferably greater than 10 kg, more preferably greater than 25 kg, more preferably greater than 50 kg, more preferably greater than 100 kg.

In accordance with a first aspect of the present invention there is provided a method for the preparation of (−)-mesembrine-besylate (compound 1B_{ES}) comprising the steps of:
Step 1:2-(3,4-dimethoxyphenyl) acetonitrile (compound 2) with THF, LiHMDS in the presence of 1-Bromo-2-chloroethane to produce 1-(3,4-dimethoxyphenyl)cyclopropane-1-carbonitrile (compound 3);
Step 2: Reacting 1-(3,4-Dimethoxyphenyl)cyclopropane-1-carbonitrile (compound 3) with DIBAL and 2-methyltetrahydrofuran to produce 1-(3,4-Dimethoxyphenyl)cyclopropane-1-carbaldehyde (compound 4);
Step 3: Reacting 1-(3,4-Dimethoxyphenyl)cyclopropane-1-carbaldehyde (compound 4) in 2-methyltetrahydrofuran and magnesium sulphate in the presence of methylamine to produce 1-(1-(3,4-Dimethoxyphenyl)cyclopropyl)-N-methylmethanimine (compound 5);
Step 4: Reacting 1-(1-(3,4-Dimethoxyphenyl)cyclopropyl)-N-methylmethanimine (compound 5) with HCl in CPME then adding methyl vinyl ketone to produce 3a-(3,4-Dimethoxyphenyl)-1-methyloctahydro-6H-indol-6-one (racemic mesembrine, compound 6);
Step 5: Reacting 3a-(3,4-Dimethoxyphenyl)-1-methyloctahydro-6H-indol-6-one (racemic mesembrine, compound 6) with of (+)-2-3-dibenzoyl d-tartaric acid in methanol and water to produce mesembrine dibenzoyl-D-tartaric acid salt (compound 1_{DBTA});
Step 6: Reacting mesembrine dibenzoyl-D-tartaric acid salt (compound 1_{DBTA}) with sodium hydroxide to produce (−)-Mesembrine (compound 1); and
Step 7: Reacting (−)-Mesembrine (compound 1) with benzenesulfonic acid in isopropyl alcohol to produce (−)-Mesembrine Besylate (compound 1_{BES}).

In a further embodiment of the invention there is provided a pharmaceutical composition comprising the product of the invention and one or more pharmaceutically acceptable excipients.

### BRIEF DESCRIPTION OF THE DRAWINGS

Embodiments of the invention are further described hereinafter with reference to the accompanying drawings, in which:

FIG. 1 shows a ¹H NMR of 3a-(3,4-Dimethoxyphenyl)-1-methyloctahydro-6H-indol-6-one (mesembrine, 6);

FIG. 2 shows a UPC of (S—S)-mesembrine (1);

FIG. 3 shows a UPC of (R—R)-mesembrine (1a); and

FIG. 4 shows a Q-NMR of (−)-Mesembrine Besylate (1_{BES})

### DEFINITIONS

Various definitions are made throughout this document. Most words have the meaning that would be attributed to those words by one skilled in the art. Words specifically defined either below or elsewhere in this document have the meaning provided in the context of the present invention as a whole and as typically understood by those skilled in the art.

The term “substantially crystalline” means at least about 50% crystalline and ranging up to about 100% crystalline. The present invention provides a salt that is at least about 50% crystalline, at least about 60% crystalline, at least about 70% crystalline, at least about 80% crystalline, at least about 90% crystalline, at least about 95% crystalline, at least about 98% crystalline, or at least about 100% crystalline in form.

The degree or percentage of crystallinity may be determined by the skilled person using X-ray powder diffraction (XRPD). Other techniques, such as solid-state nuclear magnetic resonance (NMR), FT-IR, Raman spectroscopy, differential scanning calorimetry (DSC) and microcalorimetry, may also be used.

“Subject,” “individual” or “patient” is used interchangeably herein and refers to a vertebrate, preferably a mammal. Mammals include, but are not limited to, murines, rodents, simians, humans, farm animals, sport animals and pets.

“Treating” or “treatment” of any disease or disorder refers, in some embodiments, to ameliorating the disease or disorder (i.e., arresting or reducing the development of the disease or at least one of the clinical symptoms thereof). Treatment may also be considered to include preemptive or prophylactic administration to ameliorate, arrest or prevent the development of the disease or at least one of the clinical symptoms. Treatment can also refer to the lessening of the severity and/or the duration of one or more symptoms of a disease or disorder. In a further feature, the treatment rendered has lower potential for long term side effects over multiple years. In other embodiments “treating” or “treatment” refers to ameliorating at least one physical parameter, which may not be discernible by the patient. In yet other embodiments, “treating” or “treatment” refers to inhibiting the disease or disorder, either physically, (e.g., stabilization of a discernible symptom), physiologically, (e.g., stabilization of a physical parameter) or both. In yet other embodiments, “treating” or “treatment” refers to delaying the onset of the disease or disorder.

“Therapeutically effective amount” means the amount of a compound that, when administered to a patient for treating a disease, is sufficient to affect such treatment for the disease. The “therapeutically effective amount” will vary depending on the compound, the disease and its severity and the age, weight, adsorption, distribution, metabolism and excretion etc., of the patient to be treated.

“Vehicle” refers to a diluent, excipient or carrier with which a compound is administered to a subject. In some embodiments, the vehicle is pharmaceutically acceptable.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(+)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R—S system. When a compound is a pure enantiomer, the stereochemistry at each chiral carbon can be specified by either R or S. Resolved compounds whose absolute configuration is unknown can be designated (+) or (−) depending on the direction (dextro- or laevorotatory) in which they rotate plane polarized light at the wavelength of the sodium D line. Compounds of the invention contain one or more asymmetric centres and can thus give rise to enantiomers, diastereomers, and other stereoisomeric forms, the asymmetric centres of which can be defined, in terms of absolute stereochemistry, as (R)- or (S)-. The optical activity of a compound can be analysed via any suitable method, including but not limited to chiral chromatography and polarimetry, and the degree of predominance of one stereoisomer over the other isomer can be determined.

“Enantiomeric excess” or “ee” is a measurement of purity used for chiral substances. It reflects the degree to which a sample contains one enantiomer in greater amounts than the other. A racemic mixture has an ee of 0%, while a single completely pure enantiomer has an ee of 100%. For example, a sample with 70% of one enantiomer and 30% of the other has an ee of 40% (70%-30%).

The term “enantiopure” is defined as where only one specific enantiomeric form is present.

The term “enantioenriched” is defined as when one specific enantiomeric form is present at an amount of greater than or equal to 50% of the total mixture of enantiomers, or greater than or equal to 55% of the total mixture of enantiomers, or greater than or equal to 60% of the total mixture of enantiomers, or greater than or equal to 65% of the total mixture of enantiomers, or greater than or equal to 70% of the total mixture of enantiomers, or greater than or equal to 75% of the total mixture of enantiomers, or greater than or equal to 80% of the total mixture of enantiomers, or greater than or equal to 85% of the total mixture of enantiomers, or greater than or equal to 90% of the total mixture of enantiomers, or greater than or equal to 95% of the total mixture of enantiomers.

Mesembrine is a chiral alkaloid with the CAS name: 3a-(3,4-Dimethoxyphenyl)-1-methyloctahydro-6H-indol-6-one. The structures below denote the structural configuration of the two mesembrine isomers. The enantiopure or enantioenriched mesembrine of the present invention may occur as (S—S)-mesembrine or (R—R)-mesembrine.

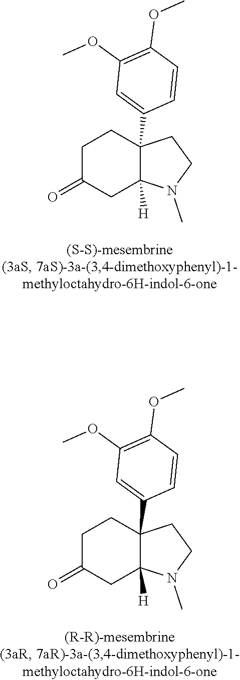 [SMILES: COc1ccc([C@@]23CCC(=O)C[C@@H]2N(C)CC3)cc1OC ; COc1ccc([C@]23CCC(=O)C[C@H]2N(C)CC3)cc1OC]

### DETAILED DESCRIPTION OF THE INVENTION

The Examples below describe the methods used to prepare racemic mesembrine and its further resolution to the two isomeric forms in high enantiomeric excess (ee).

### Example 1: Route of Synthesis to Prepare Racemic Mesembrine

Scheme 1 below denotes the synthetic route used to prepare racemic mesembrine

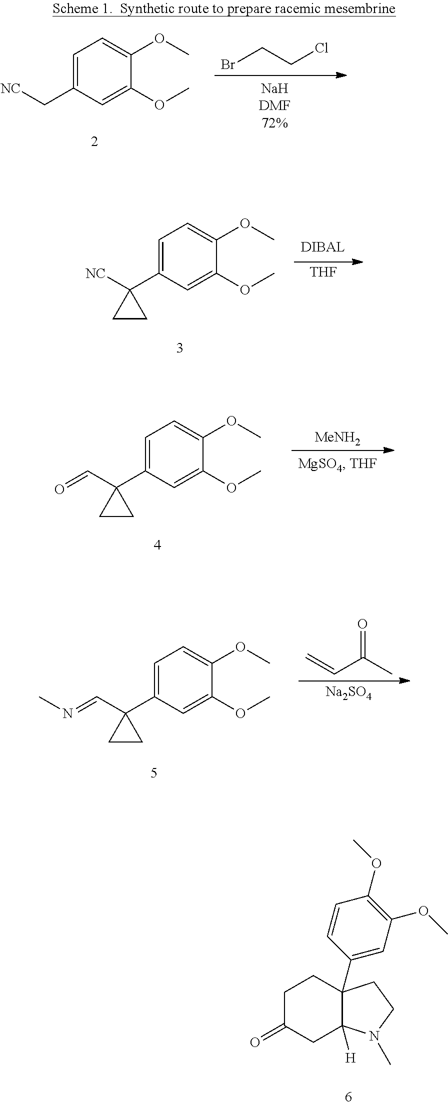

In detail the procedure for the synthesis of racemic-mesembrine (6) was as follows with reference to the numbered compounds in the scheme.

1-(3,4-Dimethoxyphenyl)cyclopropane-1-carbonitrile (3) was prepared as follows. An ice-water bath with cooled suspension of NaH (9.987 g, 60% Wt, 2.9 Eq, 0.25 mol) in DMF (20 mL, dry) was added to an ice-water bath cooled solution of 2-(3,4-dimethoxyphenyl) acetonitrile (15.001 g, 84.66 mmol) in DMF (20 mL, dry). The residual NaH was transferred using DMF (20 mL, dry). The reaction mixture was stirred at the current temperature for 1 h. 1-Bromo-2-chloroethane (24.28 g, 14.04 mL, 2.0 Eq, 169.3 mmol) was added and an exothermic reaction was observed (near immediate reflux) and the cooling bath was allowed to warm naturally.

The exothermic reaction subsided after 1 h. The reaction mixture was stirred at 30° C. external. Incomplete conversion was observed after over the weekend reaction. The conversion did not progress after adding NaH (4.015 g, 60% Wt, 1.2 Eq, 0.10 mol) and neither with adding 1-bromo-2-chloroethane (12 g, 7.0 mL, 1.0 Eq, 84 mmol). The reaction mixture was allowed to cool to room temperature and slowly added to ice-water bath cooled 1 N HCl (0.20 L). The aqueous layer was extracted with EtOAc (3×0.20 L). The combined organic layers were dried (Na2SO4), filtered and concentrated (33.015 g) containing DMF. The material was purified by automated chromatography (hep/EtOAc, 0-25%, ISCO: BKT22020060-01-08). The product containing fraction was concentrated (12.307 g) as a fluffy white solid.

1-(3,4-Dimethoxyphenyl)cyclopropane-1-carbaldehyde (4) was prepared as follows. 1-(3,4-Dimethoxyphenyl)cyclopropane-1-carbonitrile (3, 12.105 g, 59.560 mmol) was suspended in diethyl ether (300 mL, dry), cooled using a water bath and DIBAL-H in hexane (104.23 mL, 1 molar, 1.75 Eq, 104.23 mmol) was added slowly. Full addition was reached after 5 minutes. A mild exothermic reaction was observed. Full conversion was observed after 5 minutes. The reaction mixture was slowly added to water bath cooled 1 N HCl (0.30 L). The layers were separated, and the aqueous layer was extracted with EtOAc (3×0.20 L). The combined organic layers were dried (Na₂SO4), filtered and concentrated (10.383 g, 85% yield) to a white to pale yellow solid.

1-(1-(3,4-Dimethoxyphenyl)cyclopropyl)-N-methylmethanimine (5) was prepared as follows. 1-(3,4-dimethoxyphenyl)cyclopropane-1-carbaldehyde (4, 10.383 g, 50.344 mmol) was dissolved in THF (200 mL, dry). Methylamine (63 mL, 2 molar in THF, 2.5 Eq, 0.13 mol) and magnesium sulfate (29.8 g, 4.92 Eq, 248 mmol) were added. The reaction mixture was stirred overnight at room temperature using an overhead stirrer. (Near) full conversion was observed by ¹H NMR analysis. The reaction mixture was filtered over a P4-glass filter and the filtrate was concentrated (11.260 g, 99% yield) as a yellow oil containing 3% THF.

3a-(3,4-Dimethoxyphenyl)-1-methyloctahydro-6H-indol-6-one (mesembrine, 6) was prepared as follows. 1-(1-(3,4-Dimethoxyphenyl)cyclopropyl)-N-methylmethanimine (5, 21.687 g, 96% Wt, 94.945 mmol) was stripped with toluene (40 mL), dissolved in DCM (160 mL) and cooled using an ice-water bath. HCl in diethyl ether (62 mL, 2 molar, 1.3 Eq, 0.12 mol) was added and the reaction mixture was stirred for 20 minutes. The milky reaction mixture was diluted with toluene (160 mL) and concentrated. The residue was dissolved in MeCN (240 mL). Sodium sulfate (114.9 g, 8.52 Eq, 808.9 mmol) and methyl vinyl ketone (15 mL, 1.9 Eq, 0.18 mol) were added to the solution. The reaction mixture was stirred overnight at 85° C. external. The reaction mixture was cooled to room temperature. and filtered over a P3-glass filter and the residue was washed with MeCN. The filtrate was concentrated (32.740 g) as a pale red foam. The material was partitioned between water (0.10 L) and EtOAc (0.15 L). The aqueous layer was basified using 1 N NaOH (0.10 L). The layers were separated and the aqueous layer was extracted with EtOAc (3×0.15 L). The combined organic layers were dried (Na₂SO4), filtered and concentrated (27.985 g) as a red oil containing EtOAc. The material was dissolved in DCM and purified by automated column chromatography (DCM/MeOH, 0-3%). The product containing peak that was collected started from 22.5 minutes. The material was concentrated (13.866 g, 46% yield) as a red oil.

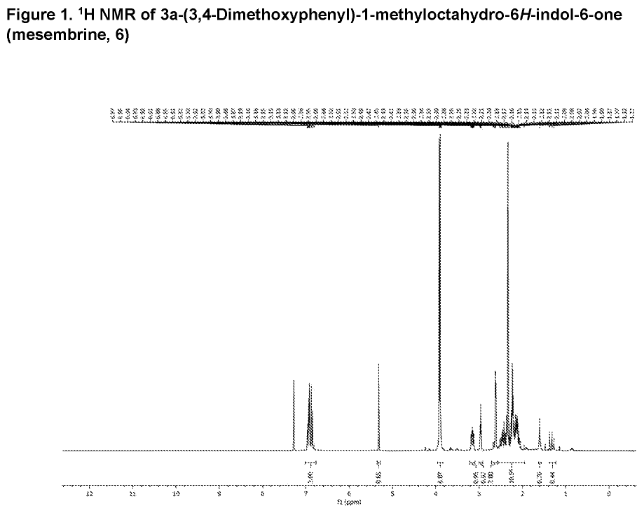

FIG. 1 shows a ¹H NMR spectra of the final racemic mesembrine material.

### Example 2: Screening of Resolution Agents to Prepare Enantiopure or Enantioenriched Mesembrine

Table 1 below describes the various chiral resolving agents that were tested in order to prepare enantiopure or enantioenriched mesembrine.

TABLE 1
Resolution screening data

| # | Resolving agent | Solvent | ee solid ^{a} | ee filtrate ^{a} | calc yield |
|---|---|---|---|---|---|
| 1 | Dibenzoyl-L-tartaric acid hydrate (1 eq) | H₂O | +12% | −42% | 77% |
| 2 | Dibenzoyl-L-tartaric acid hydrate (1 eq) | Acetonitrile | +27% | −53% | 67% |
| 3 | (R)-Phencyphos hydrate (1 eq) | H₂O | +1% | 0% | — |
| 4 | (R)-Phencyphos hydrate (1 eq) | Ethanol | +23% | −57% | 71% |
| 5 | (−)-Di-p-anisoyltartaric acid (1 eq) | H₂O | 0% | +42% | — |
| 6 | (−)-Di-p-anisoyltartaric acid (1 eq) | Acetonitrile | −28% | +75% | 73% |
| 7 | I-(−)-Di-Toluoyltartaric acid (1 eq) | H₂O | +4% | +6% | — |
| 8 | I-(−)-Di-Toluoyltartaric acid (1 eq) | Isopropanol | +6% | +14% | — |
| 9 | (R)-BINAP phosphate (1 eq) | H₂O | +1% | −69% | 98% |
| 10 | (R)-BINAP phosphate (1 eq) | Isopropanol | −30% | +54% | 64% |
| 11 | N-acetyl-L-phenylalanine (1 eq) | CPME | −65% | 0% | — |
| 12 | (S)-Naproxen (1 eq) | H₂O | +4% | 0% | 1% |
| 13 | Dibenzoyl-L-tartaric acid hydrate (0.5 eq) | H₂O | +75% | −42% | 36% |
| 14 | Dibenzoyl-L-tartaric acid hydrate (0.5 eq) | Acetonitrile | +67% | −51% | 43% |
| 15 | (−)-Tartaric acid (0.5 eq) | Isopropanol | +2% | +1% | — |
| 16 | (−)-Di-p-anisoyltartaric acid (0.5 eq) | H₂O | −27% | +62% | 70% |
| 17 | (−)-Di-p-anisoyltartaric acid (0.5 eq) | Ethanol | −11% | +43% | 79% |
| 18 | (−)-Di-Toluoyltartaric acid (0.5 eq) | H₂O | +6% | −11% | 66% |
| 19 | (−)-Di-Toluoyltartaric acid (0.5 eq) | Isopropanol | −2% | +5% | 75% |
| 20 | (−)-N-Boc-L-alanine (1 eq) | Ethanol | −3% | +1% | 28% |
| 21 | (−)-N-Boc-L-alanine (1 eq) | Acetonitrile | −2% | +1% | 38% |
| 22 | Abietic acid (1 eq) | H₂O | −5% | +4% | 42% |
| 23 | (+)-Dehydroabietic acid (1 eq) | H₂O | −1% | +3% | 86% |

^{a} A positive sign means that the material is enriched in the first eluting, enantiomer 1a. A negative sign means that the material is enriched in the enantiomer 1.

The experiment consisted of 36 different chiral resolving agents. Four of these resolving agents had two acidic moieties and were tested with 0.5 and 1 equivalents relative to racemic mesembrine 6.

Therefore, a total of 40 salts were prepared and screened against 12 different solvents, to obtain a total of 480 different experiments.

From the experiments that gave precipitates, one or two samples were filtered, and both the precipitate and the mother liquor were analyzed by chiral SFC.

Seven experiments provided significant enantiomeric enrichment in both the solid diastereomeric salt and the corresponding mother liquor.

Two resolving agents ((S)-phencyphos hydrate and(S)-(+)-1,1′-binaphthyl-2,2′-diyl hydrogenphosphate) were not further investigated due to undesirable results. (−)-Di-p-anisoyltartaric acid was not continued as the results of the crystallization with (+)-dibenzoyl-D-tartaric acid monohydrate were superior.

Surprisingly, enantiopure (−)-mesembrine (1) was able to be prepared by resolution using (+)-dibenzoyl-D-tartaric acid monohydrate.

Optimization of the resolution with (+)-dibenzoyl-D-tartaric acid monohydrate, using a two solvent system (isopropanol/water), provided a reproducible yield of 27-28% with an ee of 97.4-99.4% in a single crystallization.

The optimalisation of the solvent system is shown in Table 2 below.

| | 2-Propanol | Water | Methanol | | | Clear solution | Final |
|---|---|---|---|---|---|---|---|
| Entry | (volume) | (volume) | (volume) | e.e. (%) | Yield (%) | at reflux | temp (° C.) |
| 1 | 10 | $\frac{2}{3}$ | — | 98.7 | 22^{a} | Yes | RT |
| 2 | 10 | 10 | — | 99.7 | 26ª | Yes | RT |
| 3 | 12 | 4 | — | 98.3 | 30 | Yes | RT |
| 4 | 7.5 | 2.5 | — | 12.3 | 55 | No | RT |
| 5 | 10.5 | 3.5 | — | 98.5 | 27 | No | RT |
| 6 | 6 | 2 | 1.8 | 99.0 | 30 | Yes | RT |
| 7 | 6 | 2 | 1.8 | 97.9 | 43 | Yes | 30 |
| 8 | 6 | 2.5 | 2.5 | 29.9 | N/A | No | 30 |
| 9 | 6 | 2 | 1.8 | 99.1 | 21 | No | Reflux |
| 10 | 6 | 2 | 1.8 | 10.3 | 60 | No | 50 |
| 11 | 6 | 4 | — | 99.4 | 28 | No | 50 |
| 12 | 2.5 | 2.5 | — | 20.7 | 56 | No | 40 |
| 13 | 6 | 4 | — | 16.3 | 55 | No | 40 |
| 14 | 6 | 4 | — | 97.4 | 27 | No | 50 |
| 15 | 6 | 2 | — | 29.0 | 46 | No | 50 |

Scale: 1 mmol (entry 1); 1.7 mmol (2-7); 3.2 mmol (entries 9, 12, 14, 15); 6.3 mmol (entries 8, 10, 12). The mixtures were kept homogeneous by mechanical stirring. Heating was performed in a sand bath for a slower- and more gradual cooling process. A single crystallization was performed per entry.
ªCrystallized twice.

Promising results were obtained in entry 1 where the yield could be optimizable. This was performed by testing different solvent: co-solvent ratios as observed in entries 2 and 3. Entry 3 revealed that single crystallization could provide enantiopure material with a good yield. The total volume of the solvent system was higher than the desired 5-10 volumes and therefore the ratio was further adjusted. Entry 4 showed that those conditions were too concentrated at room temperature to obtain enantiopure material. Following this experiment, in entry 5, it was proven that high ee could be reached without fully dissolving the material.

To reduce the volume of the solvent another co-solvent was added: methanol. Entries 6 and 7 are similar with the latter being aged at 30° C. for 2 hours. This excellent result was unfortunately not reproducible with freshly prepared diastereomeric salt due to solubility issues.

The previous entries were performed with recycled material that were already exposed to several heat cycles and removal of solvent in vacuo. The insolubles were isolated at reflux in entry 9 and proved to be the desired diastereomeric salt in high enantiomeric excess with a good yield.

Several parameters were varied in entries 10-15 where 11 was the most promising, which was repeated in entry 14 to prove the reproducibility of the conditions for the resolution of mesembrine 6.

### Example 4: Resolution of Racemic Mesembrine

Scheme 2 below denotes the synthetic route used to resolve racemic mesembrine. Here compound 1 is the (S—S)-mesembrine isomer and 1a is the (R—R)-mesembrine isomer.

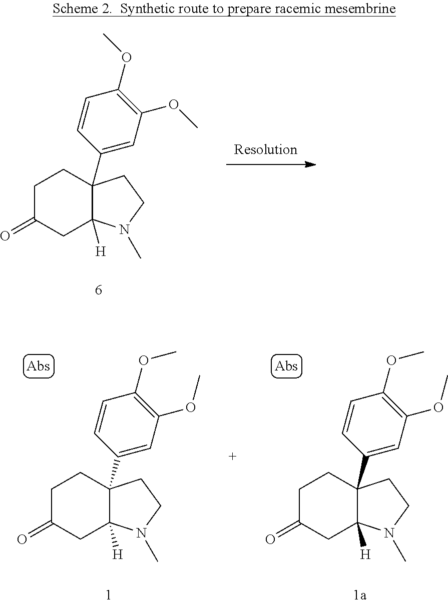 [SMILES: COc1ccc(C23CCC(=O)CC2N(C)CC3)cc1OC ; COc1ccc([C@@]23CCC(=O)C[C@@H]2N(C)CC3)cc1OC ; COc1ccc([C@]23CCC(=O)C[C@H]2N(C)CC3)cc1OC]

In detail the procedure for the resolution of enantiopure mesembrine (1, (S—S)-mesembrine) and (1a, (R—R)-mesembrine) was as follows.

The resolving agent (+)-Dibenzoyl-D-tartaric acid monohydrate (1.203 g, 1.0 Eq, 3.197 mmol) was added to a solution of racemic mesembrine (6, 1.018 g, 91% Wt, 1 Eq, 3.201 mmol) in 2-propanol (6.0 mL). Precipitation formed. Water (4.0 mL) was added, and the mixture was mechanically stirred in a sand bath. The mixture was stirred at 100° C. for 30 minutes while maintaining solids in the mixture, at 70° C. for 30 minutes, at 50° C. for 30 minutes. The mixture was filtered at 50° C. over a P4-glass filter and washed using 2-propanol (2×5 mL) to remove all colour from the solids. The solids were dried in vacuo.

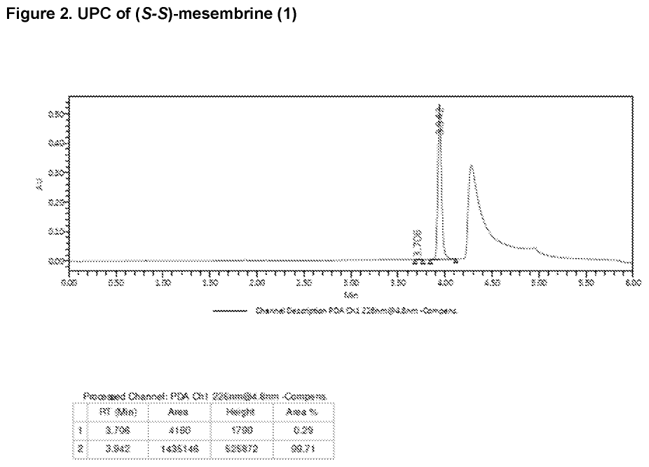

Chiral SFC showed an enantiomeric purity of 99.4% ee enriched in 1, see FIG. 2. The use of this resolution agent was able to consistently produce highly purified (S—S)-mesembrine with an optical purity of >95% ee at a high yield (>70%).

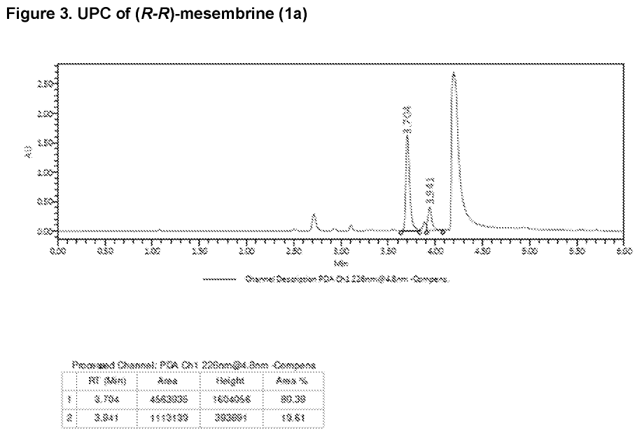

The mother liquor was dried in vacuo (1.393 g). Chiral SFC showed an optical purity of 60.8% ee enriched in 1a, see FIG. 3.

### Example 4: An Optimised Route of Synthesis to Prepare (−/−)-Mesembrine and (−/−)-Mesembrine-Besylate

The synthetic route to prepare racemic mesembrine described in Example 1 presents some limitations for scale-up and as such optimisation of the yields was undertaken in order to ensure the methodology was suitable for commercial production of mesembrine.

The preferred enantiomer of mesembrine for commercial production is (−/−)-mesembrine which may be additionally converted to a preferred salt form, mesembrine besylate.

An improved process for the preparation of (−/−)-mesembrine and it's besylate salt s described in this example.

Scheme 3 below denotes the optimised synthetic route.

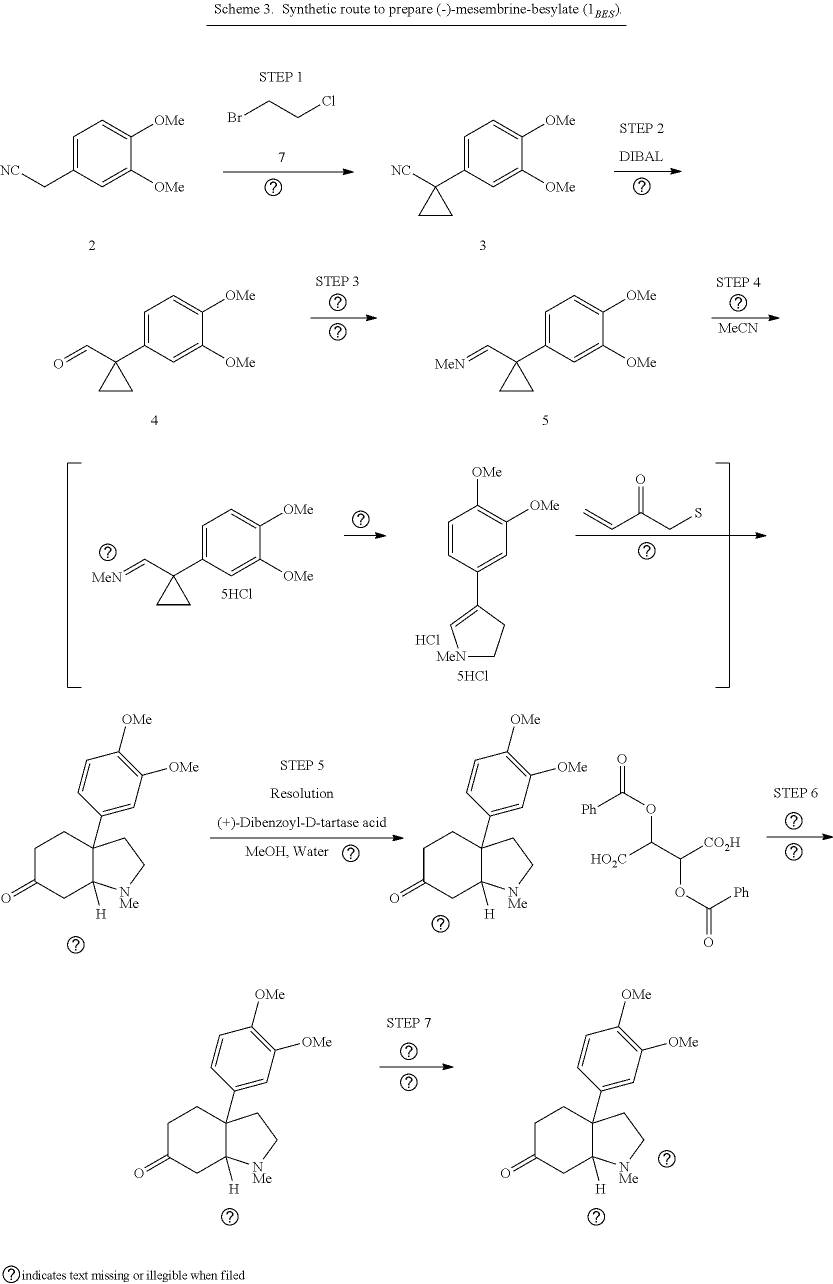

Step 1: To a 50 L reactor under N₂ atmosphere THF (10 L) and 1-bromo-2-chloroethane (2.4279 kg, 1.405 L, 2.0 Eq, 16.930 mol) were added to 2-(3,4-dimethoxyphenyl) acetonitrile (Compound 2). THF (4 L) was added to rinse the material. The mixture was cooled to −15° C. LiHMDS (24 wt % M in THF, 2.5 eq) was added slowly whilst keeping the reaction mixture between −12° C. and −16° C. Addition was complete in two hours. A brown solution was obtained which was stirred at −15° C. overnight. A sample was taken and checked by LCMS. The reaction was complete. The reaction mixture was quenched by slow addition of water (3 L). The temperature was first kept at <10° C. The mixture was then warmed up to 5° C. and more water (4.5 L) was added. The reaction mixture was then warmed to 15° C. The stirrer was stopped, and the layers were separated. The reaction mixture was washed with water/brine 5/1 (3×5 L). The brown organic layer was dried over sodium sulfate and evaporated on the rotavap at 50° C. Compound 3 1-(3,4-Dimethoxyphenyl)cyclopropane-1-carbonitrile, was obtained as an off-white/beige solid (1625 g, 94% yield).

Step 2: To a 50 L reactor under N₂ atmosphere was added to Compound 3, 1-(3,4-dimethoxyphenyl)cyclopropane-1-carbonitrile (1750 g, 1 Eq, 8.611 mol), and 2-Methyltetrahydrofuran (10.38 kg, 12.1 L, 14 Eq, 120.5 mol). The solution was cooled to −5° C. Upon cooling, the material precipitated to a yellow suspension. DIBAL-H (1.714 kg, 12.05 L, 1.0 molar, 1.4 Eq, 12.05 mol) was added slowly using a dropping funnel. Addition was complete in one hour. After the addition was complete, the reaction mixture was stirred for 2 hours at −5° C. The reaction was quenched by addition of a saturated aqueous solution of Rochelle salt (7.2 L). Warmed to 15° C. and stirred overnight. The mixture is a yellow suspension. Added 4 M HCl (11 L). A clear bright yellow mixture obtained. Stirred for 30 minutes at 17° C. The reaction was checked by LCMS and showed full conversion. The water layer was extracted with 2-meTHF (2×2 L). The combined organic layer was washed with 50% brine (2×2 L). Dried over sodium sulfate and evaporated to almost dryness. The material crystallized. Obtained 1-(3,4-Dimethoxyphenyl)cyclopropane-1-carbaldehyde, compound 4 as a beige solid (1680 g, 95%).

Step 3: To a 50 L reactor under N₂ atmosphere compound 4, 1-(3,4-dimethoxyphenyl)cyclopropane-1-carbaldehyde (3000 g, 1 Eq, 14.55 mol), 2-methyltetrahydrofuran (13.78 kg, 16.0 L, 11 Eq, 160.0 mol) and magnesium sulfate (1.751 kg, 1.0 Eq, 14.55 mol) were added. To the suspension was added Methylamine (1.130 kg, 18.18 L, 2.5 Eq, 36.37 mol, 2.0 molar solution in THF). The reaction mixture was stirred at room temperature overnight. A sample was filtered and evaporated to dryness. NMR (CDCl₃) showed full conversion. Using a Teflon tube, about half of the reaction mixture (about 18 L) was transferred out of the top of the reactor using vacuum. This yellow solution was filtered over an 8 L P2 glass filter and evaporated to dryness on the rotavap at 50° C. under vacuum. This gave a brown oil which was stored under nitrogen atmosphere at 5° C. The oil solidified to a light-yellow solid (1624 g). The other half of the reaction mixture was kept in the reactor under nitrogen atmosphere at room temperature overnight. The remaining amount of reaction mixture was filtered over an 8 L P2 glass filter and evaporated to dryness on the rotavap at 50° C. under vacuum. This gave 1770 g of (Z)-1-(1-(3,4-dimethoxyphenyl)cyclopropyl)-N-methylmethanimine (compound 5). a brown oil which was stored under nitrogen atmosphere at 5° C.

Step 4: To a 50 L reactor under N₂ atmosphere was added (E)-1-(1-(3,4-dimethoxyphenyl)cyclopropyl)-N-methylmethanimine, compound 5, (2040 g, 1 Eq, 9.303 mol) in acetonitrile (12.22 kg, 15.5 L, 32 Eq, 297.7 mol) was added. HCl (441.0 g, 4.031 L, 3.0 molar, 1.30 Eq, 12.09 mol) in CPME was added and stirred at 77° C. for 1 hour, then cooled to 50° C. Methyl vinyl ketone (832.4 g, 970 mL, 94% Wt, 1.20 Eq, 11.16 mol) was added over several minutes and the brown reaction mixture stirred at 50° C. overnight. The mixture was cooled to 20° C. 1 M aqueous HCl (9 L) and IPrOAc (8 L) were added and the layers were separated. The organic layer was extracted with 1 M aqueous HCl (1×1 L and 1×600 mL). The combined water layer was washed with IPrOAc (2×3 L). The water layer was then basified to pH 10 with 30 wt % aqueous NaOH (1700 mL). A yellow/beige suspension was obtained. The suspension was extracted with IPrOAC (1×5 L and 2×2.5 L and 1×1 L). The combined brown organic layer was washed with 50% brine (2 L), then dried over sodium sulfate and evaporated to dryness on the rotavap at 50° C. under vacuum. This gave 3a-(3,4-Dimethoxyphenyl)-1-methyloctahydro-6H-indol-6-one (compound 6) as a brown oil (1513 g, 52%).

Step 5: A flask was charged with 3a-(3,4-dimethoxyphenyl)-1-methyloctahydro-6H-indol-6-one, compound 6, (2950 g, 86% Wt pure, 1 Eq, 8.767 mol) dissolved in MeOH (15.94 L)/demi water (2.657 L). The brown solution was heated to 50° C. A solution of (+)-2-3-dibenzoyl d-tartaric acid (3.141 kg, 1.00 Eq, 8.767 mol) in MeOH (7.970 L) was added slowly over 20 min, keeping the internal temperature around 50° C. The resulting solution became a yellow/beige suspension after 5 minutes. The reaction mixture was cooled to 30° C. and stirred for 1 hour. The first heating cycle was performed. The reaction mixture was heated to reflux (67° C.) and stirred for 85 minutes. The suspension was then cooled to 30° C. and stirred overnight. Another two heating and cooling cycles (67° C. and 30° C.) were performed. The mixture was stirred again at 30° C. overnight. The suspension was heated at reflux (67° C.) for 1.5 hours), then cooled to 30° C. The suspension was immediately filtered over an 8 L P2 glass filter. The white solids were washed thoroughly with methanol/demi water (9/1, 10 L). The obtained off-white solids were dried in the oven at 50° C. for 4 days. Obtained the product Mesembrine dibenzoyl-D-tartaric acid salt (Compound 1DBTA) as an off-white solid (2600 g, 45%). The ee was 98.3%.

Step 6: A 50 L separation funnel was charged with (3aS,7aS)-3a-(3,4-dimethoxyphenyl)-1-methyloctahydro-6H-indol-6-one (2S,3S)-2,3-bis(benzoyloxy) succinate (Compound 1DBTA) (4900 g, 1 Eq, 7.565 mol) in water (34.36 kg, 34.36 L, 252 Eq, 1906 mol). To the mixture (white slurry) was slowly added sodium hydroxide (2.118 kg, 1.59 L, 30% Wt, 2.1 Eq, 15.89 mol) (pH was 10-11). The reaction mixture was kept at 20° C. (small exotherm observed) during the addition. The mixture was then stirred for another 30 minutes. The light-yellow solution was extracted with iPrOAc (1×7.5 L and 3×4 L). The organic phases were pooled together, washed with demi water (2×2.5L, then dried over Na2SO4 and concentrated on the rotavap at 50° C. under vacuum. This gave the desired product (3aS, 7aS)-3a-(3,4-dimethoxyphenyl)-1-methyloctahydro-6H-indol-6-one, (−)-Mesembrine, (Compound 1). (2113 g, 7.29 mol, 96.4% yield, 99.9% purity) as a light-yellow oil.

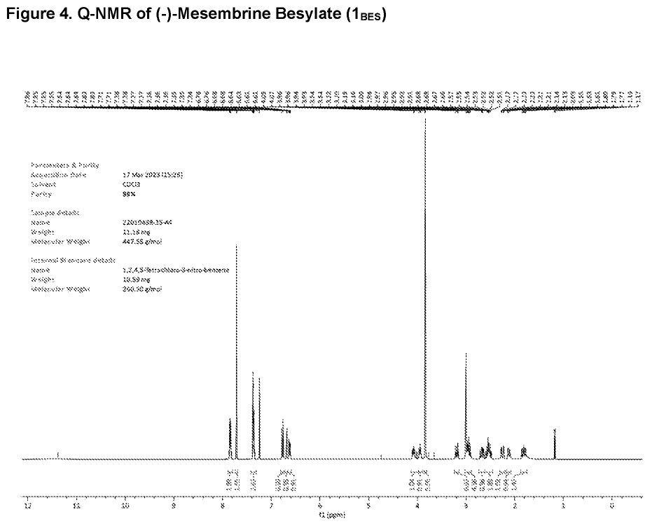

Step 7: A 50 L reactor equipped with dropping funnel and nitrogen inlet was charged with (3aS, 7aS)-3a-(3,4-dimethoxyphenyl)-1-methyloctahydro-6H-indol-6-one, Compound 1, (2110 g, 1 Eq, 7.291 mol) and Isopropyl alcohol (438.2 g, 10 L, 1 Eq, 7.291 mol) and stirred for 15 minutes. A light-yellow solution was obtained. Separately prepared a solution of benzenesulfonic acid, 98.0% (1.269 kg, 1.1 Eq, 8.021 mol) in Isopropyl alcohol was prepared (438.2 g, 5 L, 1 Eq, 7.291 mol, brown solution). A portion of the benzenesulfonic acid solution (about 1200 mL) was slowly added to the reaction mixture in the 50 L reactor. Once a precipitate started to form, the remaining of the benzenesulfonic acid solution was slowly added. The total addition time was about 1.5 hours. The obtained suspension was stirred for another 2 hours. The mixture was then filtered over an 8L P2 glass filter. The white solid was washed with 2-propanol (10 L) and dried under vacuum on the rotavap at 50° C. The product (3aS,7aS)-3a-(3,4-dimethoxyphenyl)-1-methyloctahydro-6H-indol-6-one benzenesulfonate (Compound 1_{Bes}, 3.124 kg, 6.980 mol, 95.73%) was obtained as an off-white solid. The purity was 99.8% at 215 nm and the ee was 99.1%. Q-NMR purity was 98% as shown in FIG. 4.

### CONCLUSIONS

Using the methodology of Scheme 1, racemic mesembrine was synthesized in a four-step synthesis. Such a method is superior to other synthesis methods known in the art and as such provides an improved method of preparing racemic mesembrine.

Given that there is no method in the art to prepare enantiopure mesembrine, the applicant surprisingly found that the use of (+)-dibenzoyl-D-tartaric acid monohydrate resulted in enantiopure (S—S)-mesembrine (99.4% ee) in at a yield of >70%.

Furthermore, (R—R)-mesembrine was able to be prepared with a 60.8% ee.

The advantages that invention provides is the ability to prepare substantial quantities of either racemic, enantiopure or enantioenriched mesembrine material which can be used to formulate into medicaments or for use as test agents in clinical and non-clinical studies.

There is currently no available method to prepare such pure material in high enough quantities and yields. However, the use of the current method of preparation of racemic mesembrine and the further resolution enables the preparation of kilo quantities of (S—S)-mesembrine or (R—R)-mesembrine.

An optimised route as shown in Scheme 3 was developed to enable a 7-step process to prepare (−)-Mesembrine Besylate (1_{BES}). This compound has utility as an active pharmaceutical ingredient and as such the method disclosed provides a simple and effective synthetic route that is suitable for scale-up such that kilogram quantities of the compound can be prepared.

1. A method for the preparation of 3a-(3,4-Dimethoxyphenyl)-1-methyloctahydro-6H-indol-6-one (racemic mesembrine, compound 6) comprising the steps of:
a) Reacting NaH with 2-(3,4-dimethoxyphenyl) acetonitrile (compound 2) in DMF in the presence of 1-Bromo-2-chloroethane to produce 1-(3,4-dimethoxyphenyl)cyclopropane-1-carbonitrile (compound 3);
b) Suspending 1-(3,4-Dimethoxyphenyl)cyclopropane-1-carbonitrile (compound 3) in diethyl ether and cooling in a water bath with DIBAL in hexane to produce 1-(3,4-Dimethoxyphenyl)cyclopropane-1-carbaldehyde (compound 4);
c) Dissolving 1-(3,4-Dimethoxyphenyl)cyclopropane-1-carbaldehyde (compound 4) in THF and adding methylamine and magnesium sulfate to produce 1-(1-(3,4-Dimethoxyphenyl)cyclopropyl)-N-methylmethanimine (compound 5); and
d) Stripping 1-(1-(3,4-Dimethoxyphenyl)cyclopropyl)-N-methylmethanimine (compound 5) with toluene dissolved in DCM to prepare 3a-(3,4-Dimethoxyphenyl)-1-methyloctahydro-6H-indol-6-one (racemic mesembrine, compound 6).

2. A method as claimed according to claim 1, comprising the additional steps of:
a) Adding (+)-Dibenzoyl-D-tartaric acid monohydrate to a solution of racemic mesembrine in 2-propanol; and
b) Collecting the precipitate containing an enantiopure or enantioenriched preparation of (S—S)-mesembrine.

3. A method as claimed according to claim 1, comprising the additional steps of:
a) Adding (+)-Dibenzoyl-D-tartaric acid monohydrate to a solution of racemic mesembrine in 2-propanol; and
b) Collecting the mother liquor and drying to produce enantiopure or enantioenriched preparation of (R—R)-mesembrine.

4. A method as claimed according to claim 2, wherein the yield of (S—S)-mesembrine is greater than 75%.

5. A method as claimed according to claim 3, wherein the yield of (R—R)-mesembrine is greater than 75%.

6. A method for the preparation of (−)-mesembrine-besylate (compound 1_{BES}) comprising the steps of:
Step 1:2-(3,4-dimethoxyphenyl) acetonitrile (compound 2) with THF, LiHMDS in the presence of 1-Bromo-2-chloroethane to produce 1-(3,4-dimethoxyphenyl)cyclopropane-1-carbonitrile (compound 3);
Step 2: Reacting 1-(3,4-Dimethoxyphenyl)cyclopropane-1-carbonitrile (compound 3) with DIBAL and 2-methyltetrahydrofuran to produce 1-(3,4-Dimethoxyphenyl)cyclopropane-1-carbaldehyde (compound 4);
Step 3: Reacting 1-(3,4-Dimethoxyphenyl)cyclopropane-1-carbaldehyde (compound 4) in 2-methyltetrahydrofuran and magnesium sulphate in the presence of methylamine to produce 1-(1-(3,4-Dimethoxyphenyl)cyclopropyl)-N-methylmethanimine (compound 5);
Step 4: Reacting 1-(1-(3,4-Dimethoxyphenyl)cyclopropyl)-N-methylmethanimine (compound 5) with HCl in CPME then adding methyl vinyl ketone to produce 3a-(3,4-Dimethoxyphenyl)-1-methyloctahydro-6H-indol-6-one (racemic mesembrine, compound 6);
Step 5: Reacting 3a-(3,4-Dimethoxyphenyl)-1-methyloctahydro-6H-indol-6-one (racemic mesembrine, compound 6) with of (+)-2-3-dibenzoyl d-tartaric acid in methanol and water to produce mesembrine dibenzoyl-D-tartaric acid salt (compound 1DBTA);
Step 6: Reacting mesembrine dibenzoyl-D-tartaric acid salt (compound 1 DBTA) with sodium hydroxide to produce (−)-Mesembrine (compound 1); and
Step 7: Reacting (−)-Mesembrine (compound 1) with benzenesulfonic acid in isopropyl alcohol to produce (−)-Mesembrine Besylate (compound 1_{BES}).

7. A pharmaceutical composition comprising the product of any one of claims 1 to 3 or claim 6 and one or more pharmaceutically acceptable excipients.
